(19) United States
(12) Patent Application Publication
(10) Pub. No.: US 2005/0201954 A1
(43) Pub. Date: Sep. 15, 2005

(54) Oily protective pigment dispersion for protection against uv radiation, a process for preparing it, and a cosmetic composition

(76) Inventors: Karla Araujo, Sao Paulo (BR); Nelson Luis Perassinoto, Campinas (BR); Vanessa Mendes Santos Cavalcanti, Sao Paulo (BR); Antonio Cezar Brunelli Barrozo, Sao Paulo (BR); Walter Albuquerque, Sao Paulo (BR); Andreia Cano, Jundiai (BR)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/500,756

(22) PCT Filed: Jan. 3, 2003
(86) PCT No.: PCT/BR03/00001

§ 371 Date: Apr. 8, 2005

(30) Foreign Application Priority Data
Jan. 4, 2002 (BR) PI 0200007-5

(51) Int. Cl. A61K 7/42

(52) U.S. Cl. 424/59

(57) ABSTRACT

The present invention relates to an oily dispersion of pigments for protection against UV radiation comprising, in a single base, zinc oxide and titanium dioxide added in the form of a powder, wherein the two pigments are dispersed in a single oily dispersing vehicle and the dispersion further comprises a single emollient vehicle. The invention also relates to a process for preparing said oily pigment dispersion. The dispersion of the present invention is useful to be added to cosmetic compositions, bringing about a faster way of producing compositions having a high sunshine protection factor, with a lower cost and more safety for the end products.

### FIELD OF THE INVENTION

The present invention relates to a pigment dispersion and to the process for preparing it, said dispersion being useful to prepare compositions for protection against the action of sunshine, UV radiation, for cosmetic or pharmaceutical use.

### BACKGROUND OF THE INVENTION

Pigment dispersions, such as those made of zinc oxide (ZnO) and titanium dioxide (TiO₂), available on the market, are raw materials that represent a high cost in the production of cosmetic compositions for protection against sunshine. Each pigment provides protection at a determined wavelength of the ultraviolet (UV) rays of the sun and, therefore, not only the presence of two dispersions becomes necessary, but also they should be present in suitable proportions in order to impart to the product sufficient protection against sunshine.

Inorganic pigments such as TiO₂ and ZnO are widely used in the cosmetic industry as inorganic physical sunscreens in creams and lotions designed for protection against sunshine, among other applications. Initially, the use of physical sunscreens was very limited, due to the difficulty of incorporating it in the end compositions, in obtaining effective stable products having good cosmetic appearance.

With the advance of the formulation technology, it is now possible to incorporate these filter materials in a better way, which has increased the importance of these materials in sunshine protection products and in skin care. TiO₂ and ZnO provide protection against ultraviolet (UV) radiation, the former being more effective in the UV-B region, while the latter is more effective in the UV-A region.

Since these oxides are not absorbed when applied to the skin, they are considered safer than organic filters and enable one to obtain formulations having a higher sunshine protection factor (SPF) and a wide spectrum, without the need for the presence of said organic filters. Inorganic filters attenuate the action of the sunshine radiation by combining absorption and reflection.

Protection at various wavelengths depends upon the size of the particle of active material. Therefore, one should prevent and control agglomeration of these particles, otherwise the agglomerates will have optical behavior of large particles. In other words, in the cases of inorganic filters, this represent less protection against UV rays and, consequently, a decrease in the sunshine protection factor (SPF), besides causing a whitish effect on the skin.

In order to optimize the effect of TiO₂ and ZnO, it is necessary to maintain the particles in the adequate size on the skin, also during application of the product. And one has to be sure that the particles are well dispersed in the vehicle, which is usually an emulsion.

The TiO₂ dispersions available at present may be prepared in an aqueous or oily medium, whereas the ZnO dispersions are oil based. The difficulty in having a single dispersion of these materials lies firstly in finding the dispersant and the emollient compatible with both pigments, and then the concentration of each component that will result in a stable dispersion having a high sunshine protection factor and low viscosity.

Among the documents of the state of the art that relate to sunshine protection compositions, U.S. Pat. No. 5,928,419 (Uemura et al.) may be cited as an example. This document describes the process for producing a coated organic pigment, which comprises reacting a sulfonation agent selected from sulfamic acid or a pyridine sulfur trioxide complex, with an organic pigment dispersed in a solvent, this organic pigment being soluble or partly soluble and, on the other hand, introducing a sulfonic acid group onto the surface of each particle of the organic pigment.

Further, document U.S. Pat. No. 5,527,519 (Miksits et al.) relates to a zinc oxide powder that is highly pure and finely divided, used for protection against UV in cosmetic preparations of sunshine protection creams and for daily care of the skin, as well as in varnishes and plastics. It also describes the process for preparing said ZnO powder.

Another document of the state of the art is U.S. Pat. No. 5,068,056 (Tioxide Group PLC), which teaches a dispersion of fine particles of titanium dioxide (TiO₂) containing from 20 to 60% by weight of solids and a polycarboxylic dispersant, and the use thereof in preparing cosmetic compositions for sunshine protection. The dispersions are transparent to visible light and absorb UV rays. Preferably, the largest particle size of the TiO₂ ranges from 0.01 to 0.15 micron. The dispersions are produced by grinding, preferably at high speed, until the required absorbance degree of UV rays is achieved.

Also, an organic dispersion is already known from document U.S. Pat. No. 5,468,471 (Estee Lauder), which comprises microfine TiO₂ and an organic chain without the use of any dispersing agent. The novel dispersion according to this document provides a high level of SPF, with the use of low TiO₂ contents (dry weight). Methods of preparing said dispersion and suntan cosmetic compositions comprising said dispersion are also described.

U.S. Pat. No. 6,083,490 (M&J Consultants PTY Ltd. et al) describes a process for producing a liquid dispersion of an inorganic oxide selected from zinc oxide, titanium oxide and iron oxide and having a particle size ranging from 0.02 to 30μ, the liquid consisting of or including a component having available hydrogen or oxygen ions. The liquid is selected from alcohol, ester, hydrogenated ester or polymer containing hydrogen or hydroxyl group(s). The stable dispersion may be used in topic preparations such as skin care, therapeutic, cosmetic or hair care products.

Dispersions containing TiO₂ and ZnO together on oily base are also known from the state of the art, but such dispersions do not achieve an effective result in protection against sunshine and/or do not enable one to prepare a dispersion in which the two filter components are incorporated into a stable way in the same oily base. U.S. Pat. No. 5,599,529 describes a dispersion in which TiO₂ is utilized and may or may not be coated with zinc oxide. However, according to this document, the zinc oxide is only present as a possible coating for the titanium dioxide. Therefore, this document describes, in fact, a single main filtering agent, coated with an oxide, but does not relate to two inorganic pigments (filters) added separately.

In addition, the fact that the TiO₂ particle is coated with oxides causes the wide-spectrum protection (UVA and UVB) not to be so effective. The coating with an oxide “transforms” this dioxide that has lipophilic characteristics, causing it to have more hydrophilic characteristics, which may be undesirable characteristics for the different cosmetic products. (aqueous phases and oily phases).

Dispersions containing inorganic pigments dispersed in oily phases are also described in documents U.S. Pat. No. 5,573,753 and U.S. Pat. No. 5,605,652. These patents describe a generic process for preparing a cosmetic sunscreen employing, as physical filter, a zinc oxide dispersion, which may or may not be associated to the titanium dioxide. However, even if exclusively physical filters are employed, the values of SPF obtained with that invention do not reach SPF 11, as one can see in example 3 of those documents.

The dispersions taught in these two United States patents cited above may or may not have the two filters associated, and the particles utilized are also preferably coated with one or more oxides or hydrated oxides, such as for example aluminum oxide, titanium oxide, zinc oxide, silicon oxide, magnesium oxide or zirconium oxide. As already mentioned above, this kind of coating provides some characteristics that are often undesirable in cosmetic products, since it causes the titanium oxide particles to have hydrophilic characteristics, which may not be desirable.

U.S. Pat. No. 5,573,753 foresees the possibility of using only the dispersing agent associated to the physical filter, which forms a “mass”, and then this mass is incorporated into cosmetic formulations. For each kind of desired product, each ingredient is added in its due proportion (forming bases, creams, lip-sticks, etc.).

However, although it is possible to use the same dispersant, forming a single oily base, each powdered pigment is separately dispersed and only in a second step they are mixed together, and then the other ingredients are incorporated to form the desired products. Non-use of an emollient in this first phase of the dispersion makes it difficult to homogenize this “mass” with the other ingredients that form the cosmetic products.

Thus, an objective of this invention is to obtain a stable pigment dispersion having a higher sunshine protection factor (SPF), less viscosity and the smallest particle size possible, this dispersion being prepared in a single oily base.

### SUMMARY OF THE INVENTION

The present invention relates to an oily pigment dispersion for protection against UV radiation comprising, in a single oily base, zinc oxide and titanium dioxide added in the form of a powder, wherein the two pigments are dispersed in a single oily dispersing vehicle, the dispersion further comprising a single emollient vehicle.

The invention also relates to a process for preparing said oily pigment dispersion, wherein the dispersing vehicle and the emollient vehicle are mixed in a first step to form a single oily phase, followed by a step of adding, under stirring, the TiO₂ and ZnO pigments to the oily phase obtained in the first step.

The invention further relates to a cosmetic composition comprising an oily pigment dispersion such as defined above, in association with cosmetically acceptable ingredients.

### DETAILED DESCRIPTION OF THE INVENTION

The present invention is a unique dispersion that has two main inorganic filters, also called physical filters, stabilized in an adequate proportion to obtain a high sunshine protection factor, with having better relation of protection against the UVA/UVB rays of the sun. The dispersions of the present invention provide sunshine protection factors (SPF) of about 24.

The presence of two pigments (filters) in a single oily dispersion is an innovatory and differentiating characteristic in comparison with other compositions available on the market, in which there is one dispersion for each pigment. Among other advantages, this also decreases the possibility of causing irritation to the skin.

According to the present invention, the dispersion comprises titanium dioxide (TiO₂) and zinc oxide (ZnO) in the form of a powder, used as physical filters. Preferably, the particle size of the two filters ranges from 15 to 100 nanometers.

The proportion of the filters (pigments) should be such that it will result in a stable dispersion of these two components. In a suitable way, the ratio between the pigments is of 3:1 (3 parts of TiO₂ to 1 part of ZnO), the total concentration of powders ranging from 4 to 50% by weight, based on the total weight of the dispersion. A total concentration of powders of about 40% by weight is preferred. The concentration of TiO₂ preferably ranges from 2 to 4% by weight, based on the total weight of the dispersion, whereas the concentration of ZnO ranges from 2 to 25% by weight, also based on the total weight of the dispersion. More preferably, the concentration of TiO₂ ranges from 30 to 35% by weight, and the concentration of ZnO ranges from 5 to 10% by weight, both based on the total weight of the dispersion.

As oily dispersing vehicles, polyethyleneglycol esters such as, for example, dipolyhydroxy stearate PEG 30, or silicone esters such as cetyl dimethicone copolymers may be advantageously used. Dipolyhydroxy stearate PEG 30 is preferably used.

Another component of the oily dispersion of the present invention is the emollient vehicle, which may be selected from the group comprising fatty alcohols and esters, more particularly from the group consisting of isocetyl stearoyl stearate (Ceraphyl 791), glycerol tri-2-ethyl hexanoate (Estol 3609) and propoxylated stearylic alcohol (FINSOV TN C12-15). According to a preferred embodiment of the invention, isocetyl stearoyl stearate is used. The concentration of the emollient vehicle preferably ranges from 45 to 65% by weight, based on the total weight of the dispersion, more preferably about 52%.

The incorporation of the dispersion of the invention into cosmetic products that provide protection against sunshine radiation has enabled one to decrease the number of tests to be carried out and, consequently, decrease the time it takes to develop the formulation of a sunscreen product, since the concentration of mixed filters results in a good relation of protection against UVA/UVB rays.

An advantage obtained with the above-defined oily dispersion of pigments lies in the fact that, since such a dispersion has less possibility of irritation, it may be used in a larger amount, in order to obtain a product having a higher sunshine protection factor for both grown-ups and children.

Another important factor is that, when the development of a cosmetic product is faster, there is a reduction in its cost, by virtue of a smaller amount of raw material used and shorter work time of the researcher. Of course, the return on the investment is expedited, since the end product is launched on the market earlier.

The dispersion of the present invention is prepared by a process that includes mixing the TiO₂ and ZnO pigments, the oily dispersing vehicle and the emollient vehicle. In a first step, the dispersing vehicle and the emollient vehicle are mixed to form a single oily phase. Then, the TiO₂ and ZnO pigments are added, under stirring, to the oily phase obtained in the first step. Therefore, the dispersion of the present invention is obtained by preparing a single oily dispersing base, which associates the filters and an emollient that facilitates the incorporation of this base into the end products. This facilitation is due to the addition of the pigments to the same dispersant and to the same emollient, which is the carrier that will serve for dispersion and has affinity for the two filters. The main advantage of the present invention over the state of the art is the result that is achieved when the SPF is measured, since the present invention brings about an SPF of about 24, whereas similar dispersions known from the prior art have a much lower SPF.

The illustrative examples presented below will serve to describe the invention in a better way. However, the illustrated data and procedures merely refer to some embodiments of the present invention and should not be understood as being limitative of the scope of the invention.

### COMPARATIVE EXAMPLES

In order to achieve a high sunshine protection factor, coupled with good protection against UV radiation, by using the separate dispersions, various tests were necessary, combining different amounts of each dispersion to achieve a satisfactory result.

Three experimental tests were carried out, wherein different dispersants, different emollients, and TiO₂ and ZnO in different proportions and used in isolation or in conjunction were evaluated, for the purpose of achieving a stable dispersion having a higher SPF, less viscosity and a smaller particle size.

In one of the experiments, using only one of the pigments (TiO₂), only two relatively stable compositions with regard to separation/precipitation were found. The formulation described below got a grayish coloration only after four months in the light condition, which means a somewhat higher stability of this composition in comparison with the other compositions of this experiment. The composition was as follows:

| | | % by weight, based on |
|---|---|---|
| | | the total weight of the |
| Compound | Function | composition |
| Dipolyhydroxystearate | Dispersant | About 10% |
| PEG 30 | | |
| Isocetyl stearoyl | Emollient | About 65% |
| stearate | | |
| Titanium dioxide | Pigment | About 25% |

A second experiment for determining the zinc oxide dispersion followed the same parameters as the first experiment:

| | | % by weight, based on |
|---|---|---|
| | | the total weight of the |
| Ingredient | Function | composition |
| Dipolyhidroxystearate | Dispersant | About 12% |
| PEG 30 | | |
| Isocetyl stearoyl | Emollient | About 63% |
| stearate | | |
| Zinc oxide | Pigment | About 25% |

This dispersion has presented less viscosity than the others.

However, in a third experiment, titanium dioxide and zinc oxide were used in the same oily dispersion, according to the present invention:

| | | % by weight, based on |
|---|---|---|
| | | the total weight of the |
| Ingredient | Function | composition |
| Dipolyhydroxystearate | Dispersant | 1-15 |
| PEG 30 - Arlacel P-135 | | |
| Isocetyl stearoyl | Emollient | 40-75 |
| stearate - Ceraphyl 791 | | |
| Titanium dioxide | Pigment | 2-50 |
| Zinc oxide | Pigment | 2-25 |

This kind of dispersion has presented a better result, considering all the desired characteristics of stability and sunshine protection factor.

### EXAMPLE 1

An oily dispersion was prepared with the following composition:

| | | % by weight, based on |
|---|---|---|
| | | the total weight of the |
| Ingredient | Function | composition |
| Dipolyhydroxystearate | Dispersant | 8 |
| PEG 30 - Arlacel P- | | |
| 135 ® | | |
| Isocetyl stearoyl | Emollient | 52 |
| stearate - Ceraphyl | | |
| 791 ® | | |
| Titanium dioxide | Pigment | 30.65 |
| Zinc oxide | Pigment | 9.35 |

The above composition has presented SPF 23.61, particle size of 8.8 microns, viscosity of 1,776 Cs and protection against UVA with respect to UVB of 0.76.

### EXAMPLE 2

### Preparation of the Oily Dispersion of Pigments

The oily dispersion illustrated in Example 1 was prepared as follows: the dispersant and the emollient were put in the main vessel of a Koruma equipment, then heated up to a temperature of about 45-70° C. for complete fusion of the ingredients. After the fusion, the scraper was turned on at a rotation of 20rpm, vacuum at 400 mbar and the rotor-stator (disho) was turned on at a speed of 25-40% of the maximum speed, namely 2000-3200 rpm.

The pigments were slowly added by means of an auxiliary vessel, with the aid of vacuum, the temperature being maintained at up to 62° C.

After completion of the pigment addition, the speed of the scraper was increased to about 50 rpm and the speed of the disho (rotor-stator) up to 62.5% of the maximum rotation, that is, up to 5000 rpm. Then, the mixture was stirred for a certain period of time, about 5 minutes, and the equipment was turned off.

### EXAMPLE 3

### Cosmetic Formulations Comprising the Pigment Dispersion Illustrated in Example 1

A - Photoprotective Cosmetic Composition with SPF 30

| | % by weight, based on the total |
|---|---|
| Ingredient | weight of the composition |
| Dipolyhydroxystearate PEG 30 | 4.0 |
| Silicone 345 | 4.0 |
| Isohexadecane | 8.0 |
| Oily Vitamin E | 2.0 |
| Candelilla wax | 0.5 |
| Pigment dispersion | 40 |
| Sodium chloride | 0.5 |
| Tetrasodium EDTA | 0.3 |
| Glycerin | 5.0 |
| Pigment | 1.0 |
| 2-Bromo-2-nitropropane | 0.01 |
| Demineralized water | 29.69 |

B - Photoprotective cosmetic composition with SPF 30

| | % by weight, based on the total |
|---|---|
| Ingredient | weight of the composition |
| Dipolyhidroxystearate PEG 30 | 2.0 |
| Silicone 749 | 2.0 |
| Arlacel 987 | 1.0 |
| Oily Vitamin E | 0.5 |
| Candelilla wax | 0.5 |
| Dermol 89 | 0.5 |
| Alkoline MCM | 1.0 |
| Candelilla wax | 0.5 |
| Pigment dispersion | 40 |
| Cetiol OE | 4.0 |
| Iodine propenyl butyl carbamate | 0.2 |
| Demineralized water | 37.95 |
| Phenoxyetanol | 1.0 |
| MgSO₄ | 0.7 |
| Disodium EDTA | 0.1 |
| Glycerin | 3.0 |
| Silicone VS 7158 | 3.0 |

C - Cosmetic composition of the type water-in-oil
emulsion for use in make-up (corrective base)

| | % by weight, based on the total |
|---|---|
| Ingredient | weight of the composition |
| Dipolyhydroxystearate PEG 30 | 2.0 |
| BHT | 0.025 |
| Cyclomethicone | 3.0 |
| PPG-15 stearyl ether | 3.0 |
| Isoparafin | 12.0 |
| Pigment dispersion | 40 |
| Yellow iron oxide | 2.0 |
| Red iron oxide | 0.4 |
| Magnesium sulfate.7H₂O | 0.7 |
| Dipropyleneglycol | 3.0 |
| Methylparaben | 0.2 |
| Propylparaben | 0.1 |
| Imidazolydinyl urea | 0.3 |
| Demineralized water | 100 |

1. An oily dispersion of pigments for protection against UV radiation, characterized by comprising, in a single oily base, zinc oxide and titanium dioxide added in the form of a powder, wherein the two pigments are dispersed in a single oily dispersing vehicle and the dispersion further comprises a single emollient vehicle.

2. The oily dispersion according to claim 1, characterized in that the ratio between the pigments of TiO₂ and ZnO is 3:1.

3. The oily dispersion according to claim 1, characterized in that the total concentration of powders in the dispersion ranges from 4 to 50% by weight.

4. The oily dispersion according to claim 3, characterized in that the total concentration of powders in the dispersion is of 40% by weight.

5. The oily dispersion according to claim 1, characterized in that the concentration of TiO₂ ranges from 2 to 40% by weight, based on the total weight of the dispersion.

6. The oily dispersion according to claim 5, characterized in that the concentration of TiO₂ ranges from 30 to 35% by weight.

7. The oily dispersion according to claim 1, characterized in that the concentration of ZnO ranges from 2 to 25% by weight, based on the total weight of the dispersion.

8. The oily dispersion according to claim 7, characterized in that the concentration of ZnO ranges from 5 to 10% by weight.

9. The oily dispersion according to claim 1, characterized in that the particle size of the TiO₂ and ZnO pigments used ranges from 15 to 100 nanometers.

10. The oily dispersion according to claim 1, characterized in that the dispersing vehicle is selected from the group consisting of polyethyleneglycol and silicone esters.

11. The oily dispersion according to claim 10, characterized in that the dispersing vehicle is dipolyhydroxy stearate PEG 30.

12. The oily dispersion according to claim 1, characterized in that the emollient is selected from the group consisting of isocetyl stearoyl stearate, glycerol tri-2-ethyl hexanoate and propoxylated stearylic alcohol.

13. The oily dispersion according to claim 1, characterized in that the emollient is used in a concentration ranging from 45 to 65% by weight, based on the total weight of the dispersion.

14. A process for preparing an oily dispersion as defined in any one of claims 1-13, which comprises mixing TiO₂ and ZnO pigments, an oily dispersing vehicle and an emollient vehicle, characterized by comprising a first step of mixing the dispersing vehicle and the emollient vehicle to form a single oily phase, followed by a step of adding, under stirring, the TiO₂ and ZnO pigments to the oily phase obtained in the first step.

15. A cosmetic composition characterized by comprising a dispersion as defined in any one of claims 1-13 in association with cosmetically acceptable ingredients.
